(19) United States
(12) Patent Application Publication
(10) Pub. No.: US 2017/0325780 A1
(43) Pub. Date: Nov. 16, 2017

(54) STAT LINE CENTRAL LINE MEDICAL KIT

(71) Applicant: Luis Ferreira Maracaja NETO, San Antonio, TX (US)

(72) Inventor: Luis Ferreira Maracaja NETO, San Antonio, TX (US)

(73) Assignee: THE BOARD OF REGENTS OF THE UNIVERSITY OF TEXAS SYSTEM, Austin, TX (US)

(21) Appl. No.: 15/520,021

(22) PCT Filed: Oct. 28, 2015
(86) PCT No.: PCT/US15/57804

§ 371 Date: Apr. 18, 2017

Related U.S. Application Data
(60) Provisional application No. 62/069,814, filed on Oct. 28, 2014.
(60) Provisional application No. 62/114,499, filed on Feb. 10, 2015.

(51) Int. Cl. A61B 8/00; A61B 8/08; A61B 46/20; A41D 13/12; A61M 25/00

(52) U.S. Cl. CPC A61B 8/4422; A61M 25/002; A61B 8/4218; A61B 46/20; A41D 13/1209; A61B 8/0841

(57) ABSTRACT

Certain embodiments are directed to materials and devices to be used in conjuction with interventional medical procedures. In certain aspects the interventional procedure is ultrasound guided venous cathterization.

This application claims priority to U.S. Provisional Patent Application Ser. No. 62/069,814 filed Oct. 28, 2014 and 62/114,499 filed Feb. 10, 2015, each of which is incorporated herein by reference in its entirety.

### BACKGROUND

Certain embodiments are related to the field of medicine particularly related to interventional procedures such as ultrasound guided procedures or surgery.

Millions of venous catheterization procedures are performed daily. When peripheral venous access can not be easely obtained, ultrasound guidance is used for peripheral or central access. Central venous catheterization is a medical procedure with potentially serious complications. The use of ultrasound for real time imaging guidance remarkably reduces these complications and has become the standard of care for central line catheterization.

A sterile field and sterile precautions are needed when performing central line placement in order to avoid line infection and other serious complicaitons with high morbidity. Central venous catheter placement is unsuccessful in 20 percent of attempts with an associated 10 percent minor and major complication rate (e.g., arterial puncture, iatrogenic pneumothorax, hemothorax, air embolus, arrhythmias, catheter knotting, hemothorax, chylothorax, and brachial plexus injury). Also, catheter-related bloodstream infections are common, costly, and potentially lethal. Each year in the United States, central venous catheters may cause an estimated 80,000 catheter-related bloodstream infections and, as a result, up to 28,000 deaths among patients in intensive care units (ICUs).

In current clinical practice the material needed to perform this procedure are packaged separately. To conduct ultra sound guidance an ultrasound machine is needed for imaging. The procedure involves several steps and check lists have been created in order to provide the appropriate materials for this placement of a central line. These steps are time consuming and extra personal is frequently needed to handle necessary supplies for the line placement. Difficulties in having all the materials and sufficient personel are excerbated because central line placement is frequently needed in emergency situations. All the steps and time needed to provide appropriate sterile field and sterile ultrasound probe increase the time needed to perform the procedure.

Time limits frequently lead to several problems such as, non compliance with use of ultrasound, contamination of sterile material, lack of time for planning the best approach to insert the needle towards the target, and consequentially a less precise placement. One or more of these issues increase of number of attempts and increase the risk for complications.

There remains a need for more efficient material and imaging assistance devices that minimize the time and personnel needed for ultrasound guided line placement.

### SUMMARY

Certain embodiments described herein are directed to a disposable kit designed for ultrasound guided procedures. The kit provides all the necessary supplies for a procedure and organizes those supplies in an as needed order. The design of the kit reduces the need of physical space for both storage and use, as well as localizing all components in one location. The kit reduces amount of manipulation need for opening packages and accessing supplies, which reduces the potential of infection, as well reducing the number of personell need to prepare for a procedure. The kit provides for sterile preparation of the provider, the equipement, and the patient all in one kit. Furthermore, the design of the kit provides for efficient ergonomics as components of the kit opens around the equipment used for guiding the procedure and provides the instruments needed in a readily accessible location when those instruments are needed. In additional embodiments a mount/support arm is provided or used that will hold an ultrasound transducer allowing the physician to have hands free to perform the procedure. The kit design and peripherals save time and increase physician compliance with the use of ultrasound protocol.

Embodiments of the invention include materials and devices to be used in conjuction with interventional medical procedures, such as ultrasound guided venous cathterization. Certain embodiments are directed to accessory materials and kits for providing a sterile surface, sterile components (including instruments and sterile dress), and an environment for medical procedures using ultrasound guidance.

In certain aspects a sterile kit or package described herein includes the materials needed to perform a central line placement. The kit is ergonomically designed to minimize risk of infection, minimize personnel required, and minimize time for insertion of central line catheters. In certain aspects the sterile kit is designed to be used in conjunction with an ultrasound transducer. The kit is initially enclosed in a storage bag in order to maintain sterility of the kit. The storage bag can be removed and discarded when the kit is needed. The kit comprises a shell or container that surrounds an ultrasound transducer cover, a surgical drape, and an instrument package. The shell or container can be a plastic box or flexible bag that can be sterilized and sealed, enclosing certain kit components and maintaining sterility of the external surface of the transducer cover. The kit is configured to provide access to the lumen or interior of the transducer cover while maintaining a sterile seal and sterile environment for the remainder of the kit.

The ultra sound transducer cover is a flexible sleeve having a closed end and an open end. The transducer cover can be folded or package such that an ultrasound transducer can be positioned at the closed end of the cover prior to opening the shell or container. Once the shell or kit is open the transducer cover is extended along any support or cables attached to the transducer. The cover surface to be placed against a patient is sterile while the internal surface of the cover is not necessarily sterile.

The kit shell or container has a first compartment or a preparation compartment that is accessible when the kit shell or container is intact. This first or preparation compartment contains items needed to initiate the intervention process, prepare the patient prior to intervention, and/or prepare the person performing the intervention. In certain aspects the first compartment is accessible by a removable cover once the storage bag is removed, while the ultrasound transducer cover, a surgical drape, and an instrument package remain sealed in the shell or container. The first or preparation compartment contains preparative items such as hat, mask, gown, gloves and skin preparation solution(s), e.g., ChloroPrep®. Typically the first or preparation compartment is opened after the kit is removed from the storage bag and the sterile dress is employed followed by the use of the patient preparation supplies. In certain aspects the shell or cover is configured so that the ultrasound transducer can be positioned inside the transducer cover prior to opening the shell or container. The sterile kit is designed to be used in a certain sequence and can reduce the time needed to perform line placement while allowing the entire procedure to be performed in a sterile fashion by one health care provider.

Once the patient and provider are prepared and the transducer positioned in the transducer cover, the kit shell or container is opened to access the surgical drape and instrument package. The surgical drape is package in such a way so that the transducer can be positioned with respect to the patient and the surgical drape deployed to form a covered field. Once the surgical drape is deployed the instrument package and be removed or detached and placed on the surgical drape. The instrument package is configured to have one or more sealed compartments that can be accessed by removing or peeling a cover to a compartment. In certain aspects the linear order of the instruments correlates with the steps involved in the intervention procedure.

Thus, to initiate an intervention procedure one would need to obtain the kit, remove the storage bag and open the preparation compartment. Once the preparation compartment is opened the person dresses and prepares the patient. Once these preparations are complete, or while these preparations are being performed, the ultrasound transducer is positioned in the transducer cover. Once the transducer is positioned the kit is opened and the surgical drape and instrument package is accessed. The drape and instrument package positioned for initiation of the intervention procedure. Once the line is placed and secure the kit components can be thrown away.

Certain embodiments are directed devices and systems incorporating the devices to support objects during a medical procedure. In certain aspects a support is configured to hold or position an ultrasound probe. The support maintains an object in a stable position allowing the physicians to have both hands free once the object is in position so as to perform other necessary tasks. In one embodiment the rigidity of the support is controlled by touch sensor or a mechanical button or switch so that the rigidity is controlled by the physician positioning the support. In certain aspect the support is flexible when the sensor, switch, or button is engaged by the physician and rigid when sensor, switch, or button is released. Once released the support maintains its position. In certain aspect the support device allows fast and precise positional adjustments in six degrees of freedom. The support can also be used to support ultrasound transducers for continuous monitoring and diagnostic purposes such as continuous echocardiography. In certain aspects the support further comprises a laser level aligned with the ultrasound transducer. The laser level provides a contiguous line with the ultrasound beam allowing very precise guidance during needle insertion.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of an ultrasound machine having an transducer.

FIG. 2. Illustration of a transducer positioned to be inserted in one embodiment of an unopened Stat line kit.

FIG. 3. Illustration of a transducer being fully inserted into one embodiment of a Stat line kit.

FIG. 4. Illustration of one embodiment of a Stat line kit being opened after the transducer is inserted, and sterile dressing and preparation items have been removed from the preparation compartment.

FIG. 5. Illustration of one embodiment an open Stat line kit being positioned on a patient.

FIG. 6. Illustration of one embodiment of a Stat line kit being deployed.

FIG. 7. Illustration of one embodiment of a fully deployed Stat line kit ready for intervention procedure.

FIG. 8. Illustration of one embodiment of an ultrasound transducer coupled to a support arm.

FIG. 9. Illustration of one embodiment of a support arm.

FIG. 10. Illustration of a cross section view of one embodiment of a support arm.

### DESCRIPTION

Embodiments described herein allow fast, precise adjustments in positioning imaging devices and for proving guidance or proper placement of central venous catheter (CVC), also known as central line, in an environment that reduces the risk of infection. A central line is a catheter placed into a large vein in the neck, chest, or groin. A central line is used for administering medication or fluids, obtaining blood samples, hemodynamic monitoring, temporary cardiac pacing, and/or performing hemodialysis. Devices and materials described herein can be used by interventional radiologists, surgeons, anesthesiologists, emergency personnel, and critical care physicians. Embodiments of the kits and devices described herein provide the supplies needed to perform the procedure in a single package. The design of the kit provides for a package opening process that allows for performance of all necessary steps in sterile fashion. The kits and device minimize personal, as well carts and tables.

The disposable kit described herein can be used with the support described herein or any other positioning or mount devices that would hold the probe and the sterile material preventing mechanical fall with gravity. The disposable kit described herein does not require a table or surface to support the kit during opening or manipulation of the drape or the other components. The kit with all the supplies are positioned around the support, with the supplies being located in different sterile compartments that will be opened in multistep fashion according to the procedure needs.

### I. STAT Line Kit

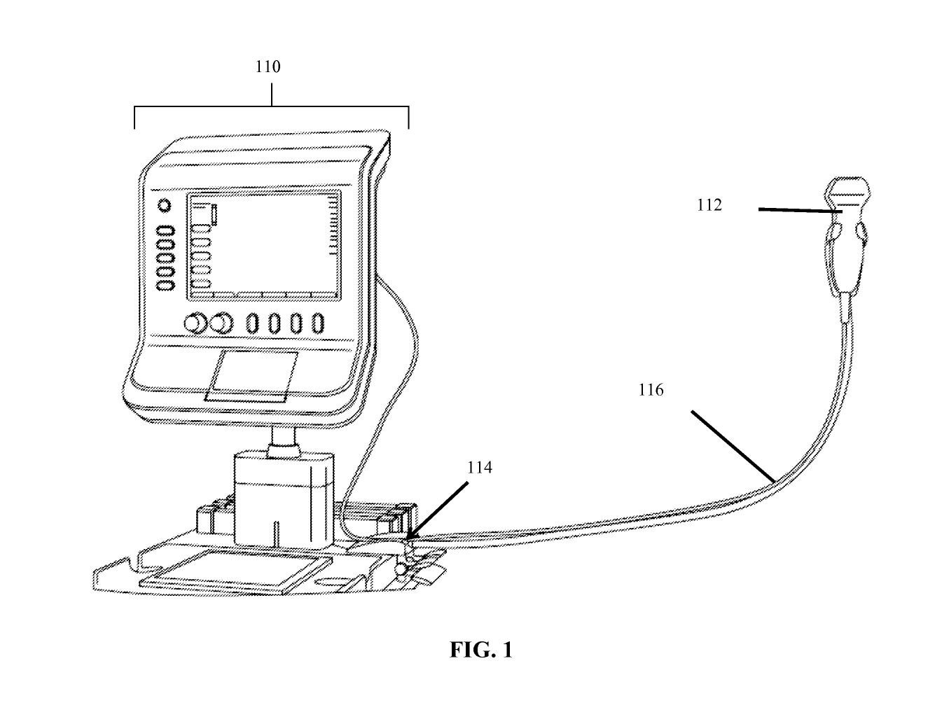

Pre-sterilized medical procedure kits are known and used for various medical procedures. Such sterilized procedure kits are provided with a plurality of components used in connection with a particular surgical procedure. Certain embodiments are directed to sterilized surgical kits organized and accessed in a way that maintains a sterile environment or reduces the risk for infection during a procedure, as well reducing the time for initiating and/or performing the procedure. Kits of the invention can be used in conjunction with ultrasound devices and for performing ultrasound guided procedures. One example of an ultrasound device is illustrated in FIG. 1. FIG. 1 shows an ultrasound machine 110 coupled to an ultrasound transducer 112 via a transducer support arm 116.

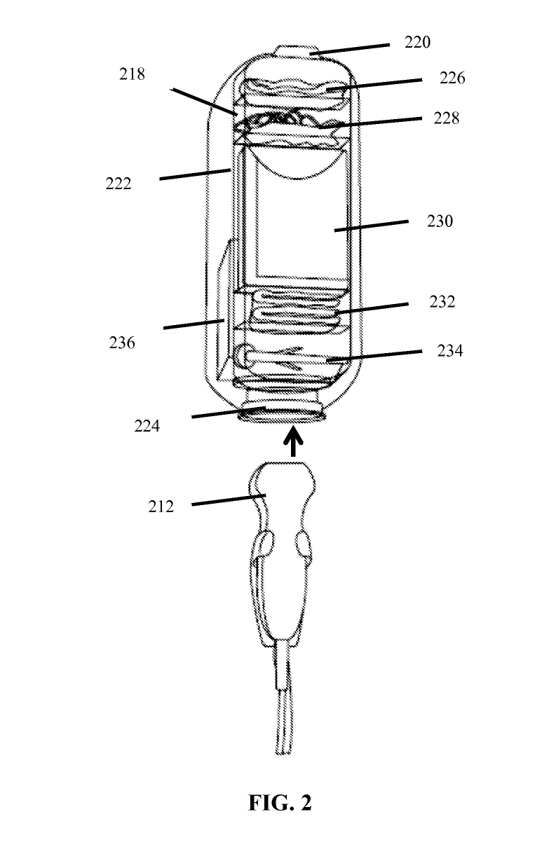

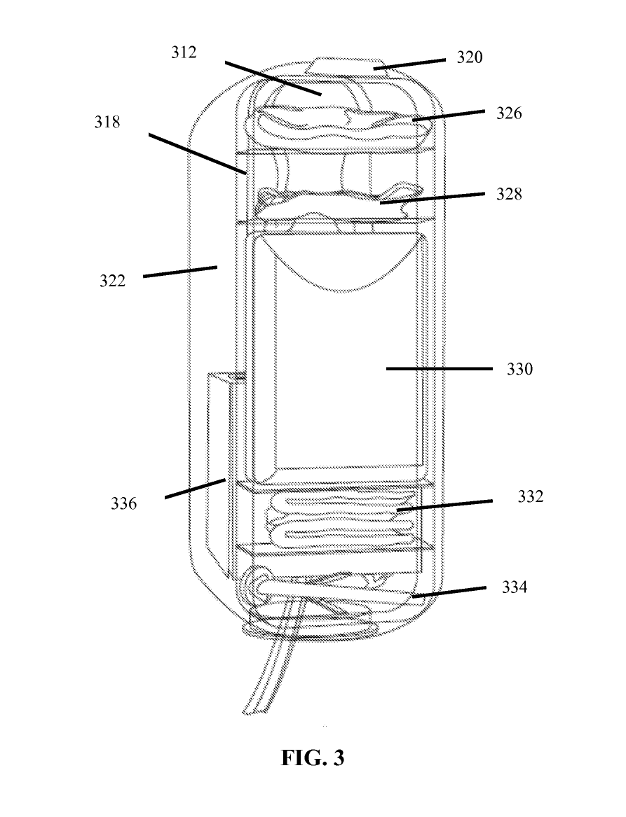

The kit can be provided enclosed in a storage bag or covering that can be removed prior to using the kit. One example of a kit is provided in FIG. 2. The kit shown in FIG. 2 has been removed from the storage bag. The kit can include a drape 236, surgical instruments (folded in drape 236), and transducer cover 224 contained by a shell or container 222. In certain aspects the drape 236 is configured with an opening for an ultrasound transducer. The opening is also configured to provide access for insertion of a needle or other device while imaging with ultrasound. The opening can be configured with a sterile transducer cover 224 attached. The shell or container 222 can have a preparation compartment 218 attached or formed by shell 222. The preparation compartment 218 can be configured with a removable cover 220. The removable cover 220, when removed, provides access to sterile dress and preparation items. The preparation compartment can include a sterile hat 226, sterile mask 228, sterile gown 230, sterile gloves 232, anticeptic solutions, and eye protection (not shown in FIG. 2) that is contain with the sterile, sealed preparation compartment 218 that is configured to be accessed when need for the kit arises. The removable cover 220 can be detachable coversheet with a sterile inside surface that is in contact with other sterile kit components and a non-sterile outside surface. The shell or container 222 contains access port to allow insertion of the ultrasound transducer 212 into the ultrasound cover 224 prior to unpacking of the kit. In certain aspects the kit can also comprise sterile compartments or packages containing other devices and materials needed for a surgical procedure. In one embodiment an instrument package is folded with drape 236 so that the instrument package is accessible once the drape 236 is deployed. The drape 236 in a folded in a configuration that can be unfolded to cover a portion of the patient once the transducer is properly positioned. FIG. 3 illustrates the transducer 312 fully inserted into shell or container 322. Also shown in the foreground is the preparation compartment 318 with cover 320 intact maintaining the sterility of the compartment components 326-334.

Prior to using the kit and intitiating the procedure any auxiliary equipments or devices will need to be assembled and/or prepared. In certain aspects an ultrasound device with its transducer will need to be acquired and prepped for use. In certain aspects a support device as described herein can be used in conjunction with an ultrasound device to provide a stabilized holder for an ultrasound probe. In certain aspect an ultrasound conducting material is positioned inside and outside of the transducer cover in order to enhance ultrasound transmission to the transducer. The material that will brought in contact with the patient can be provided in sterile compartment that can be opened just prior to use in order to maintain sterility for as long as possible.

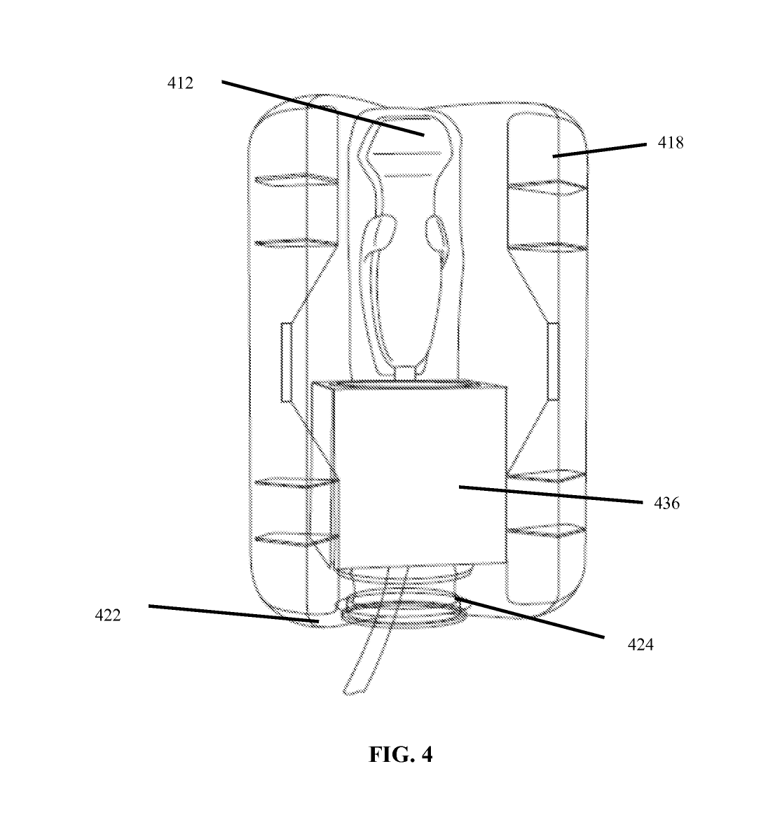

Once the ultrasound device and the transducer are properly positioned the shell or container is removed. FIG. 4 illustrates one embodiment of a kit that has shell 422 opened exposing the drape 436 and the transducer 412 positioned in the transducer cover 424. Also shown in the preparation compartment 418 that has been accessed by removing the cover and accessing the sterile dressing and preparation items. In certain aspects the preparation compartment can be attached to the kit by an adhesive or clasp and is detached prior to opening shell or container 422.

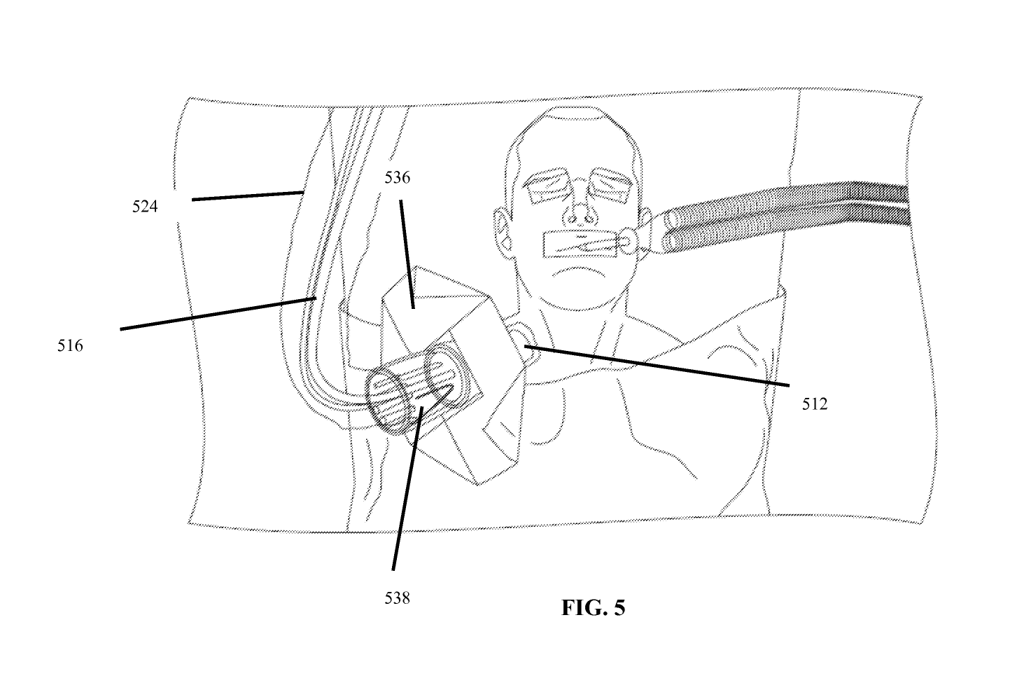

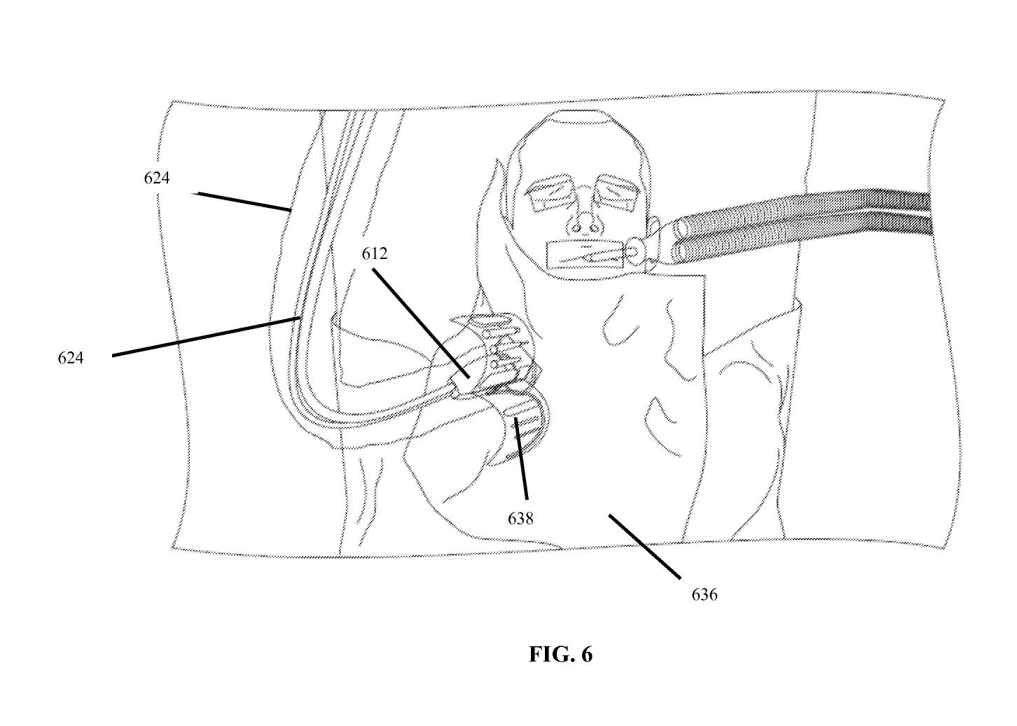

As illustrated in FIG. 5, once the kit is accessed the transducer 512 in the transducer cover 524, which has been extended along the transducer support arm 516 is placed in position over the patient in the appropriate position. The ultrasound probe is positioned and in some embodiments is locked in place using support arm 516. When the drape 536 is deployed instrument package 538 becomes accessible. FIG. 6 depicts the detachment of instrument package 638 from transducer 612.

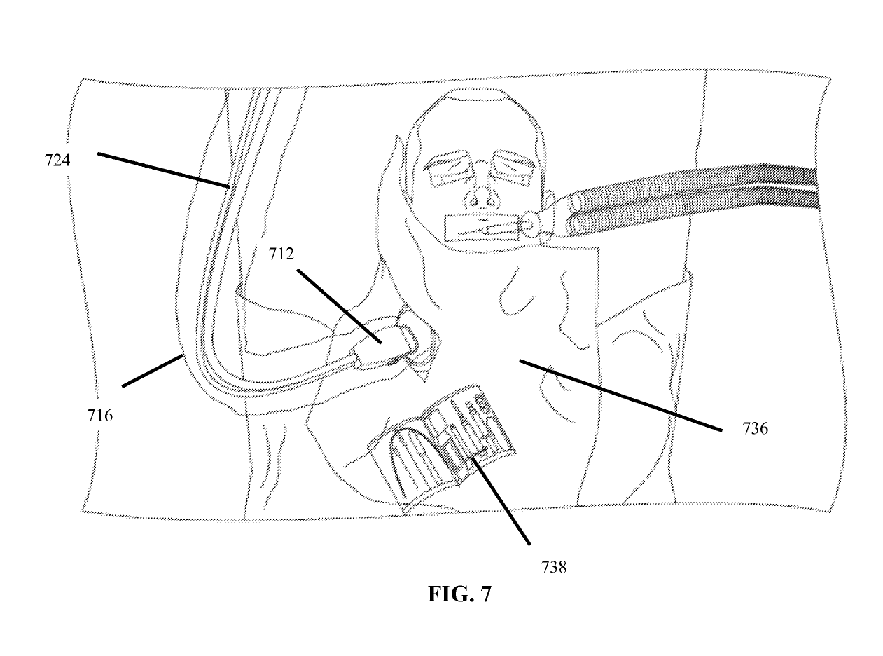

As shown in FIG. 7, when the ultrasound is appropriately positioned instrument package 738 containing the necessary supplies for the central line line placement is detached, placed on drape 736, and opened to access intervention supplies. The instrument package 738 will be configured to be inside the drape 736 when the drape is folded for packaging of the kit. Once the kit is accessed and the drape 736 deployed the instrument package 738 will be positioned in a convenient location relative to the opening for ultrasound transducer. The ultrasound is used to guide placement of central line, the placement finalized and secured.

The instrument package can contain one or more surgical or medical tools necessary to perform a particular medical or surgical procedure. This can be most any type of surgical or medical equipment or tools such as, for example, needle(s), cannula(s), sponge(s), wipe(s), sutures, forcep(s), scissor(s), and scalpels of various configurations. Other non-limiting examples of supplies or tools include sponges, syringes, anesthetics, needles, clamps, cannulas, vials, gauze pads, swabs, stapling devices, dissectors, and sutures to name a few.

### II. Positioning Device

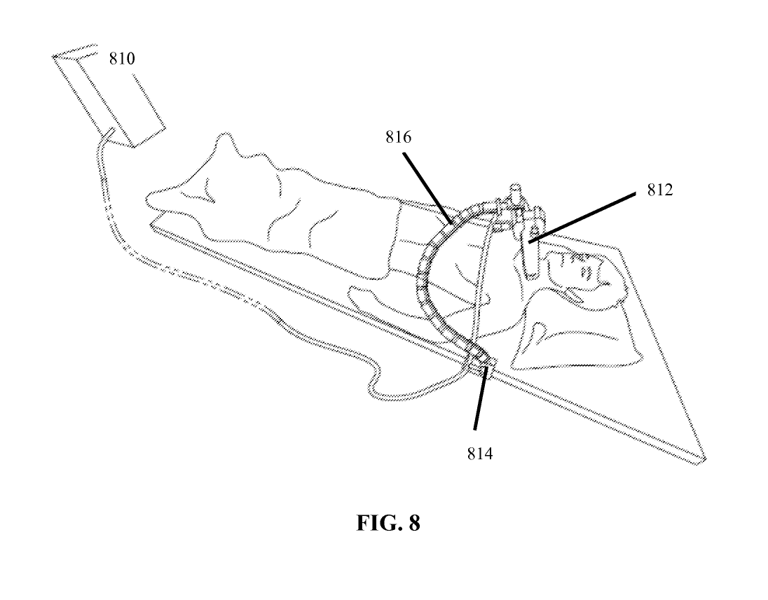

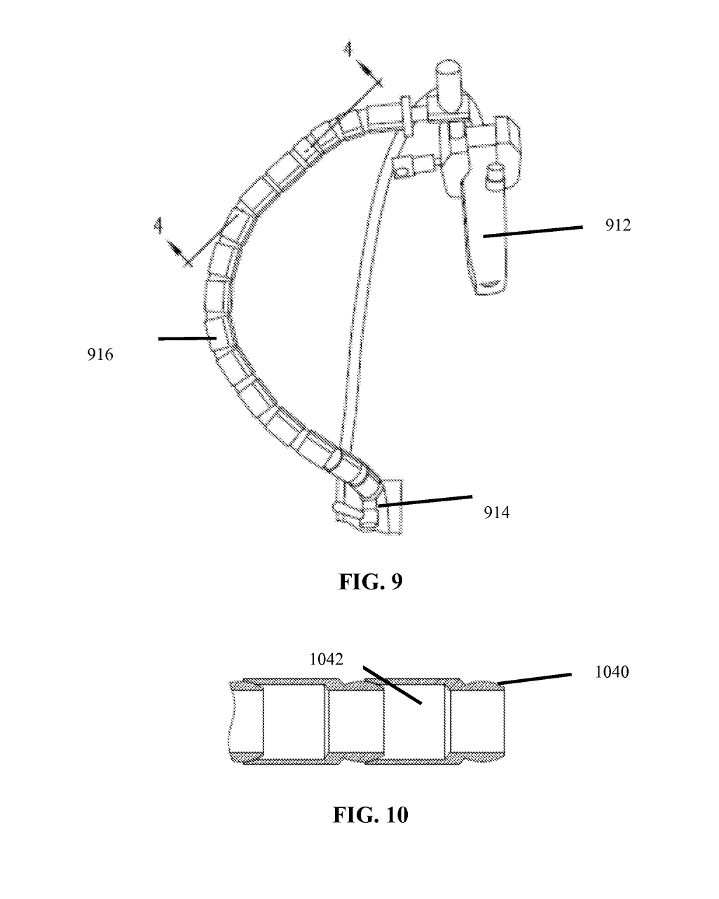

The following includes a description of a positioning device, related components and material, and methods of employing the device. FIGS. 8, 9, and 10 illustrate components and uses for a positioning device as described herein. A support to assist in guiding the placement of central lines can comprise a support arm 816 and 916 having a proximal end, a distal end, a plurality of joints along the length of the arm, and a stabilizing mechanism or mount 814, 914 coupled to an actuator, wherein (i) the arm is configured to flex around the joints (see FIG. 10 for an example of one embodiment of the joint mechanism) when the stabilizing mechanism is disengaged and configured to be rigid when the stabilizing mechanism is engaged, (ii) the proximal end is configured to attached to a structure, and (iii) the distal end is configured to be attached to an attachment (such as transducer 812, 912). In certain aspects the arm 816, 916 is a cylindrical component having a series of at least two interconnected inner links (two links form a joint) providing flexibility to the arm. In certain aspects the arm is about 30, 60, 120, 150, 200, 300 or more cm in length, including all values and ranges there between, along the long axis, and about 1, 2, 3, 4, 5, 6, 7, 8, 9, 10 cm, including all values and ranges there between, in outer diameter. In certain aspect the arm forms an inner channel. In a further aspect the inner channel can have a diameter of about 0.5, 1, 2, 3, 4, 5, 6, 7, or 8 cm, including all values and ranges there between. The arm can be made from a polymer, metal, or a combination thereof. The links provide the ability to provide the arm in a flexible configuration or a rigid configuration. The arm can be manipulated while in the flexible configuration to a needed shape and position. Once in the need shape and position the arm can be rendered rigid to hold the shape and position. Pressure can be applied to each of the joints to increase friction between each joint component, immobilizing the joint. The pressure can be applied by tightening a connecting component between the base and the proximal end, or a position between the base and proximal end, of the arm. An arm extends from the distal end and includes a series of relatively moveable outer links that define a channel that runs along the long axis of the arm. In certain aspects the channel is configured for disposal of the connecting component. Each outer link has a proximal mating surface and a distal mating surface configured such that the connecting component is engage able to transform the arm from the flexible configuration to the rigid configuration. The connecting component pulls the mating surfaces together resulting in fixation of the arm in a selected orientation.

In certain embodiments the connecting component is connected to the proximal end of the arm and at a second point in the distal portion of the arm. The connecting component is configured to be disposed or run along the all or part of the length of the arm. In certain aspects the connecting component is a polymer or metal cable, or a polymer or metal chain. The connecting component is configured so that tension on the connecting component is transferred to the joint of the arm. In certain aspects the proximal end of the connecting component is attached at a proximal point near before the first joint of the arm, and the distal end of the connecting component is attached after the last joint of the arm. In certain aspects the proximal connection is proximal to last joint in the arm. In certain aspects the connecting component is disposed with a channel that runs along the long axis of the arm. In a further aspect the connecting component can run along the outer surface of the arm. In still a further aspect the connecting component can be positioned in a slot or groove along the exterior surface of the arm.

In certain embodiments the arm includes a connecting component that is configured to provide tension on the arm by forcing two or more consecutive joints together. Thus, the arm can have a low tension state where it is flexible and the joints in the arm are freely rotatable; and a high tension state where two or more joints are forced together so that the arm is rigid and there is no movement of the joints under tension. The tension can be modulated by an actuator. The actuator can be used to apply or release tension on the connecting component. The connecting component can be moved axially by the actuator. The actuator can move the connecting component and apply tension to joints of the arm. The application of tension results in a fixed and/or rigid conformation of the arm. Release of the tension by the actuator releases tension from connecting component and results in a flexible conformation of the arm.

The actuator can be adjusted (engaged or disengaged) by a screw or a reel mechanism that can lock and unlocked the actuator from a tension providing position. If the actuator comprises a screw mechanism it will comprise an internal member that is threaded with the proximal or distal end of the interior cavity of the arm such that the connecting component can be pushed/pulled for releasing or applying tension, respectively, on the arm by advancing or withdrawing the actuator along a threaded portion. A reel mechanism can include a cable reel having a cable (i.e., a connecting component) extending from the reel, the proximal end of the cable being attached to a reel and the distal end of the cable being attached to the arm. In certain embodiments the reel is positioned on the distal end of the arm and the distal end of the cable attached proximal end of the arm. In other embodiments the reel is positioned on the proximal end of the arm and the distal end of the cable attached distal end of the arm. In certain aspects the reel can be placed anywhere along the arm and may comprise two cables one attached to the proximal end of the arm and a second cable attached to the distal end of the arm. The reel can be a spring-loaded rotating spool that winds or unwinds the cable to either apply or release tension on the cable. The reel can be configured with a one touch locking system where a user can engage a mechanism to keep the reel from moving. In instance where the lock is engaged while tension is being provide to the cables, the arm will be locked in a rigid conformation.

In certain aspects the cable reel is a rotating spool that winds or unwinds the cable. In certain aspects the cable reel includes a toothed housing configured for receiving the end of a cable. In certain aspects the spool is contained within the housing. The housing having a knob or trigger configured to wind or unwind the cable around the spool. The knob or trigger, and the spool assembly cooperate to engage the spool with a ratchet feature for winding the spool when the knob or trigger is turned in one direction or depressed to tension the cable, and for releasing the spool to release tension on the cable. The cable may be a nylon coated or stainless steel cable.

The arm extends from a first end and includes a series of movable links or joints. The interior of the links or joints defines an axial bore or channel extending along arm. The connecting component can be disposed within the channel. The arm is movable in one or a plurality of planes, directions and/or degrees of freedom, and may be rotated, bent, or twisted about the long axis of the arm.

In certain aspects a link or joint includes a distal portion of a first arm segment and the proximal portion of an adjoining segment. In certain aspect a first end can have a spherical configuration and a second end can have a cylindrical configuration that receives the spherical portion. This configuration provides articulation at each link or joint. In certain aspects the receiving portion is movable in one or a plurality of planes, directions and/or degrees of freedom, may be rotatable or twisted and/or axially movable with respect to the spherical portion. In certain embodiments the arm may have a circular cross section. In other aspects the arm may have an oval, oblong, triangular, rectangular, square, polygonal, irregular, uniform, non-uniform, variable and/or tapered cross section. The arm comprises a series of links joints to provide flexible articulation and orientation of arm, and a stabilization mechanism to fixed fix the arm in a selected orientation for support and positioning of an instrument.

The proximal end of the positioning device can comprising a mounting member 114, 814, 914 (e.g. a clamp) for attaching a device to a support. The mounting member can be connected to an adjustable clamp, a bed rail, a table, a cart, or a device. In certain aspects the mounting member provides for attachment to an ultrasound device.

In certain aspects an attachment (e.g., transducer 112-912) is attached to the distal end of the arm. The attachment can be directly coupled (integrated with the arm) or indirectly coupled via an adjustable clamp or other mechanism that can be used to remove or attach an attachment. In certain embodiments the attachment is an imaging probe. In a further aspect the attachment is an ultrasound transducer.

1. A surgical kit comprising a transducer cover, a drape, and an instrument package enclosed in a shell or container that is removably connected to the transducer cover, the kit is configured such that an internal surface of the transducer cover is accessible from the outside of the shell or container for insertion of a transducer into the transducer cover while maintaining sterility of the external surface of the transducer cover, the drape, and the instrument package, wherein the drape is folded in a manner to allow deployment when needed and the instrument package is removably connected to the transducer cover, the drape, or both the transducer cover and the drape.

2. The kit of claim 1, further comprising a preparation compartment containing sterile dress and items for preparing for a surgical procedure, the preparation compartment having a removable cover forming sterile seal.

3. The kit of claim 2, wherein the sterile dress comprises a sterile hat, sterile mask, sterile gown, and sterile gloves.

4. The kit of claim 2, wherein the items for preparing for a surgical procedure are antiseptic and sterilizing solutions.

5. The kit of claim 1, wherein the instrument package contains at least one a needle, a cannula, a sponge, a wipe, sutures, a forcep, a scissor, or a scalpel.

6. The kit of claim 1, wherein the surgical kit is a kit for vascular intervention.

7. The kit of claim 6, wherein the kit for vascular intervention is a central line installation kit.

8. The kit of claim 1, wherein the transducer cover comprises an ultrasound gel at the interface between the transducer cover and the transducer, and/or on the external surface of the transducer cover opposite the transducer head.

9. A support device comprising:
an arm having a proximal end, a distal end, a plurality of joints along the length of the arm, and a stabilizing mechanism, wherein (i) the arm is configured to be flex around the joints when the stabilizing mechanism is disengaged and rigid when the stabilizing mechanism is engaged, (ii) the proximal end is configured to attached to a structure, and (iii) the distal end is configured to be attached to an attachment.

10. The device of claim 9, wherein the stabilizing mechanism is a cable and reel actuator.

11. The device of claim 9, wherein the arm is operatively coupled to an ultrasound device.

12. The device of claim 9, wherein the attachment is an ultrasound transducer.

13. An ultrasound system comprising the support device of claim 9 operatively attached to a ultrasound system.
